(19) United States
(12) Patent Application Publication
(10) Pub. No.: US 2003/0204196 A1
(43) Pub. Date: Oct. 30, 2003

(54) Arthroscopic and endoscopic knot tying device and method of use

(76) Inventor: Edward S. Bittar, Indialantic, FL (US)

Correspondence Address: ROGER M. RATHBUN, 13 MARGARITA COURT, HILTON HEAD ISLAND, SC, 29926, US

(21) Appl. No.: 10/194,802

(22) Filed: Jul. 12, 2002

Related U.S. Application Data
(60) Provisional application No. 60/375,390, filed on Apr. 24, 2002.

(51) Int. Cl. A61B 17/04

(52) U.S. Cl. 606/148

(57) ABSTRACT

A device and method to aid the tying of a suture knot to repair tissue of a patient. The device is an elongated body with a distal end and a proximal end. An end opening is formed in the distal end that is just slightly larger than a suture strand. A passageway is formed between that end opening and a side opening in the side wall of the device. One of the free suture strands is passed through the distal opening to be accessible at the side wall opening. A knot is tied in the free ends of a suture strand. The device is used to push the knot tightly against the tissue since the knot is larger than the distal opening. The physician can pull on the free ends of the suture to tighten the knot, and readily secure the knot tightly against tissue in a relatively inaccessible location within the patient.

### REFERENCE TO RELATED CASES

The present application is based upon Provisional Application Serial No. 60/375,390 filed Apr. 24, 2002 and entitled ARTHROSCOPIC AND ENDOSCOPIC KNOT TYING DEVICE AND METHOD OF USE.

### BACKGROUND OF THE INVENTION

The present invention relates to a device for facilitating the carrying out of a medical procedure and, more particularly, to a device and method to assist in the tying of a suture knot to secure tissue of a patient.

There are, of course, many surgical procedures that require suturing of the tissue of a patient to close an opening in the patient and/or reattach tissue to bone or other soft tissue of the patient. In the majority of cases, the strands of the suture are tied by the physician at a fairly convenient location, that is, the actual suture knot is accomplished at a site external of the patient and, therefore, well accessible by the physician.

On the other hand, there are a great number of medical procedures that require the tying of suture strands together where the location of those strands is at an inconvenient location, such as within the body of the patient, and thus the task of tying together the suture strands, already a difficult task, becomes exceedingly more difficult since the physician must access the suture strands well within a remote location within the body of the patient. In such cases, it is still important to fully tighten the knot of the suture strands to adequately close the particular tissue together even though the site is difficult to access so as to bring the tissue together in a tight, secure relationship. In effect, it is necessary to locate the knot tightly against the tissue so that the suture can pull the tissue together with the knot securely held against that tissue.

One of such locations where the tying of a suture knot is considerably difficult, due to the inaccessibility of the site of the suture knot, is in the carrying out of arthroscopic and/or endoscopic procedures.

Thus, while the present invention will be described with respect to an arthroscopic surgery, particularly performed on the shoulder or knee of a patient, it will be seen and understood that the present invention can be equally applicable to any location within a patient wherein the site at which the suture knot must be located is relatively inaccessible.

Turning, therefore to arthroscopic surgery, for the purpose of explaining the present invention, a common injury to the shoulder of a patient is a tearing of the rotator cuff, which can occur, for example, as a result of some overhead use of the upper extremity. The rotator cuff tear can cause progressive shoulder pain, swelling, weakness, and dysfunction. The dysfunction can increase the longer the tear remains in the rotator cuff, and it is therefore necessary to take some action to repair the tear for the wellbeing of the patient.

Accordingly, some of such tears can be corrected by surgery, particularly arthroscopic surgery where a fiber optic instrument is utilized to aid the physician in observing the procedure carried out within the patient's shoulder and so as to cause a minimum of trauma and invasion of the shoulder while carrying out the procedure. With the surgery, the procedure is basically to suture the torn portion of the rotator cuff to thereby repair the tear and to reconstitute the rotator cuff back to its original status.

Obviously, even with an arthroscopic instrument, the operation is a difficult, delicate procedure, requiring great skill since it requires the use of a suture or sutures that cross or span the tear and then a knot formed and tightened to bring the individual strands of the suture together so as to pull separated sides of the tear together. Since, however, the knot that is tied to bring the tissue together must ultimately be secured tightly against the tissue, it is quite difficult to tie that knot such that it is pushed against the tissue where the tissue itself is located within such a relatively inaccessible location within the patient's shoulder such that the location must be observed through an arthroscope in order for the physician to even be able to view the injury and tend to its correction.

Additionally, the meniscus of the knee, a fibrocartilage shock absorber, can be torn by excessive compressive and torsional loading of the meniscus. The two segments of a torn meniscus can be repaired using the device described in a fashion that is similar to the repair of the rotator cuff. The operation and a new device to carry out the operation is shown and described in co-pending U.S. patent application Ser. No. 10/000,000 entitled Meniscus and Soft Tissue Repair Device and Method, filed on the same day as the present application and the disclosure of which is incorporated herein by reference.

Also, suturing tissues in other body spaces, that include abdominal, thoracic or pelvic cavities, is extremely difficult because of the inaccessibility of the recesses of deep body cavities. Laparoscopic surgery, fiber optic surgery of a body cavity, permits visualization of the recesses of the cavity and the structures contained therein. The device described can be used in a similar fashion to suture and repair tissues in body cavities under laparoscopic visualization

Accordingly, it would be advantageous to have a device that can facilitate the aforedescribed medical procedure to carry out the positioning of a suture knot that is tightly located against the tissue that is being sutured where that tissue is located in a relatively inaccessible area within the patient.

### SUMMARY OF THE INVENTION

Accordingly, the present invention relates to a device and a method to make a knot in suture strands such that the knot can be located tightly against the particular tissue that is being sutured despite the location of that tissue in a relatively inaccessible location within a patient.

With the present invention, there is a main body having an elongated central axis and having a distal end and a proximal end. The body can be a solid rod having a narrow or small diameter with an opening at the distal end of the main body that preferably is formed to be located in the distal end itself in alignment with the elongated central axis. The distal end also is comprised of a smooth round shape that allows it to be introduced atraumatically into the patient with a small skin incision.

A passageway is formed in the body of the device, and which extends inwardly of the distal opening and terminates at an opening formed in the side wall of the main body. The passageway comprises an initial portion that extends inwardly of the distal opening coaxially along the central, elongated axis of the main body with a transition portion that extends from that initial, coaxial portion of the passageway to the opening in the side wall of the main body.

The transition portion is preferably an arcuate surface so that a suture strand can be introduced into the distal opening and be pushed along the passageway to provide a smooth transition as the direction of the travel of that strand is changed as it moves from the coaxially aligned portion through the transition portion and then emerges outwardly through the opening in the side wall of the main body. As therefore can be understood, the coaxially aligned portion of the passageway does not continue further inwardly within the main body beyond the transition portion so that the suture strand is not impeded from smoothly being passed or threaded completely through the passageway to emerge outwardly from the side wall opening where the suture thread can be readily accessed by the physician.

Once the suture strand has been passed through the two areas of the tissue that are desired to be brought together, one of those free ends is threaded through the passageway by entering the strand into the opening in the distal end of the device and pushing that suture strand through the passageway until it emerges at the opening in the side wall of the device where it is accessible to the physician carrying out the procedure.

Thus, in the use of the method of the present invention, a sliding knot can be initially tied in the free ends of the suture strand that has been passed through the tissue of a patient by a physician in carrying out the normal suturing of the tissue. The sliding knot is of the type that allows the knot to slide along the length of one of the free ends of the suture strand.

The free end of the suture strand that emerges through the side opening can then be grasped by the physician and the device used to push and slide the knot along the other free end of the suture strand until the knot is seated tight against the tissue. That sliding action is made possible by the predetermined dimensions of the distal opening so that one strand of suture can freely move through that distal opening but the knot is too large to enter the distal opening, thus, in effect, the opening is just larger than the single strand of suture but too narrow for a knot to pass therethrough.

Accordingly, once the knot is seated against the tissue, tension can be applied to the free end of the strand of suture that passes through the passageway in the device and the suture loop tightened as the tension is applied.

Further, the suture loop of the knot is tightened as additional tension is applied alternately to both free ends of the suture strands while the knot is being held in the tight relationship with the tissue by the distal end of the device. Additional knots can thereafter be placed in the suture strands until a secure knot is obtained that is tightly seated against the tissue. The excess suture strands can then be trimmed, leaving a secure knot held tightly seated against the tissue despite a relatively inaccessible location with the patient.

These and other features and advantages of the present invention will become more readily apparent during the following detailed description taken in conjunction with the drawings herein.

### BRIEF DESCRIPTION OF THE DRAWINGS

FIG. 1 is a side view of the knot tying device of the present invention;

FIG. 2 is a front view of the device of FIG. 1;

FIG. 3 is a side cross sectional view of the device of FIG. 1;

FIG. 4 is an enlarged end view of the device of FIG. 1;

FIG. 5 is a perspective view of the knot tying device of the present invention;

FIG. 6 is a schematic view showing a step in a procedure to secure a suture knot against the tissue of a patient;

FIG. 7 is a schematic view of a further step in the procedure of FIG. 6; and

FIG. 8 is a schematic view of a further step in the procedure of FIG. 6.

### DETAILED DESCRIPTION OF THE INVENTION

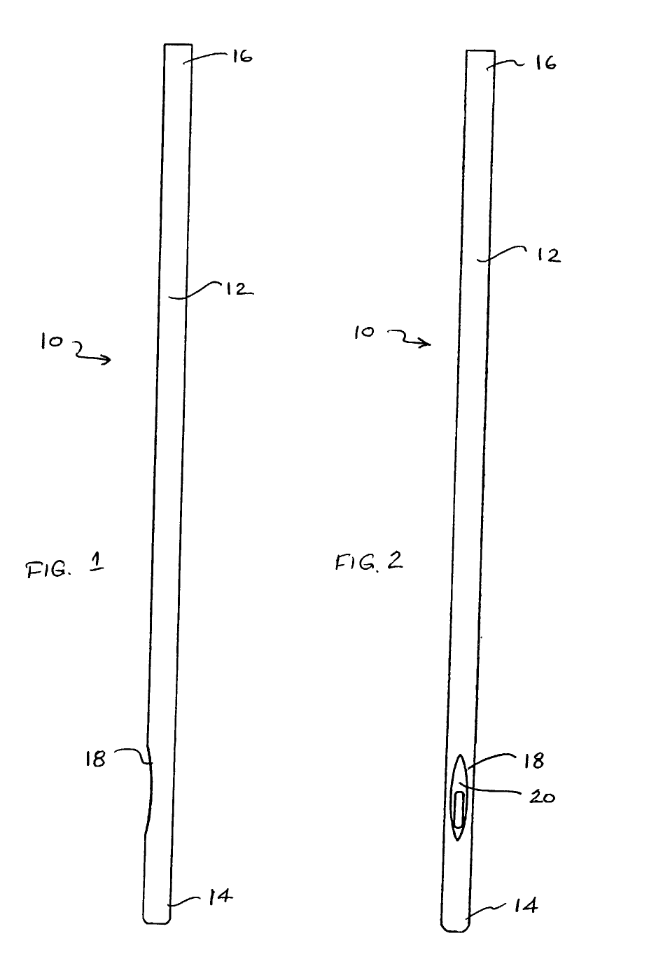

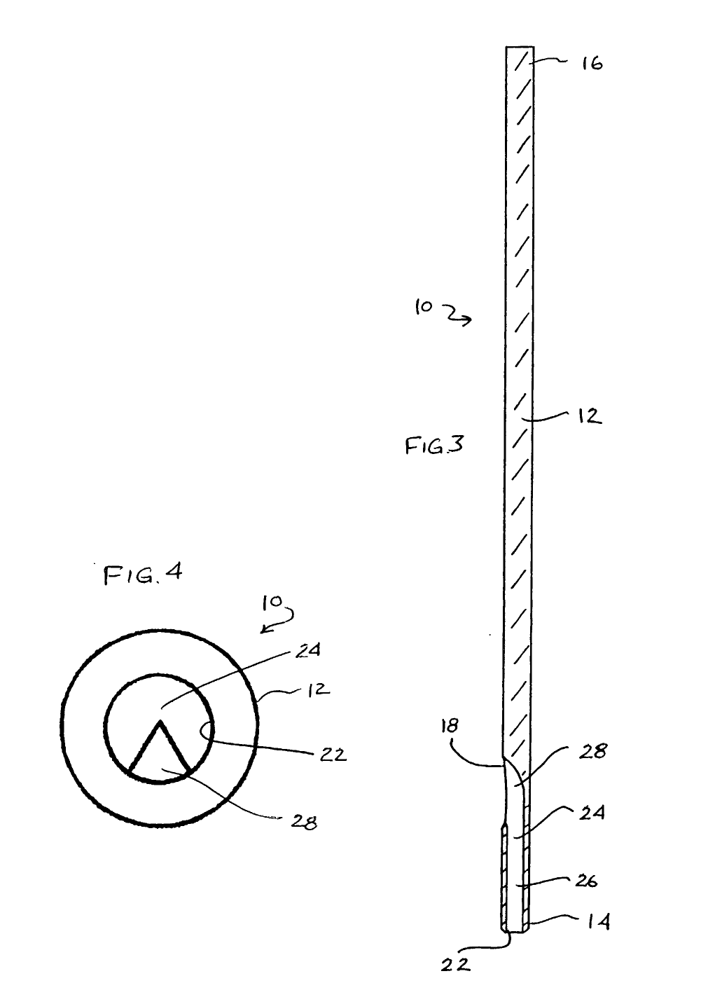

Referring now to FIGS. 1 and 2, there is shown a side view and a front view, respectively, of a knot tying device 10 constructed in accordance with the present invention. In the FIGS., there can be seen that the device 10 is basically a solid rod 12 having a distal end 14 and a proximal end 16. The solid rod 12 is preferably constructed of a metal material that can enable the physician to carry out the sterilization of the knot tying device 10 after its use, however, the knot tying device 10 can also be constructed of a plastic material or other disposable material to enable the device to be made of a relatively inexpensive material so as to be disposable after each use.

The distal end 14 is formed in a rounded shape so that the knot tying device 10 can be introduced atraumatically into the body of a patient through a small skin incision, such as would be used in introducing the knot tying device 10 into a joint of a patient. Preferably, the overall diameter of the solid rod 12 is about 5 millimeters or less for use with small joint recesses, however, the diameter can be larger for larger body cavities.

There is a side opening 18 formed in the side wall of the solid rod 12 and in the embodiment shown, there is an arcuate recessed area 20 in that side wall that is provided in the formation of the side opening 18.

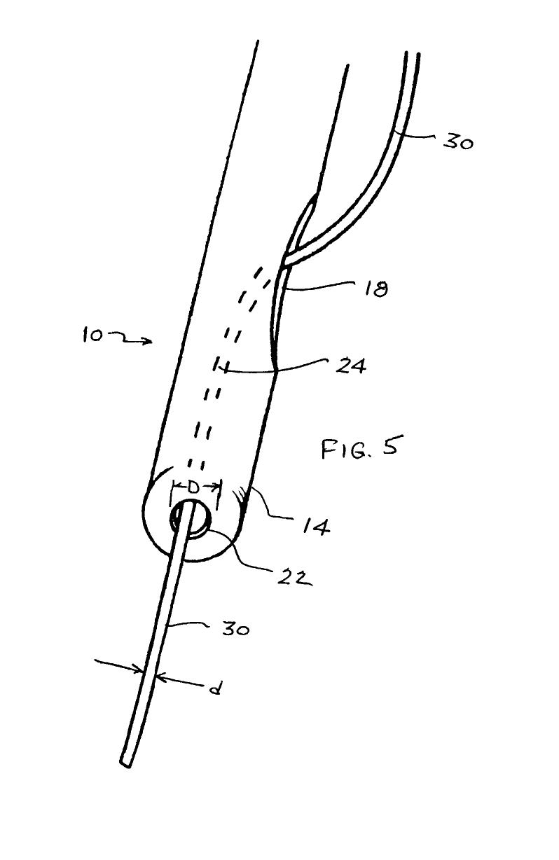

Turning to FIG. 3, there is shown a side cross-section view of the knot tying device 10 and showing the side opening 18 and also shown an end opening 22 that is formed in the distal end 14 of the knot tying device 10. As can be seen, the overall solid rod 12 has an elongated longitudinal axis and the end opening 22 generally is in alignment with that elongated axis. The overall length of the solid rod 12 can be about 30 centimeters for use in endoscopic surgery, however, the length can be longer, such as 90 centimeters or more for other surgeries in other areas of the patient.

A passageway 24 is formed in the solid rod 12 extending inwardly from the end opening 22 and initially, there is a relatively straight portion 26 of that passageway 24 that is coaxial with the elongated, longitudinal axis of the solid rod 12. There is also a transition portion 28 of the passageway 24 that changes the direction of the passageway 24 so that it emerges at the side opening 18.

As can be seen, specifically in FIG. 3, the transition portion 28 is an arcuate internal surface of the solid rod 12 and the transition portion 28 widens as it forms the side opening 18, as shown particularly in FIG. 2. Preferably, the side opening 18 is relatively close to the distal end 14 and can be about 20 to 40 millimeters from that distal end 14.

Turning briefly to FIG. 4, there is shown an end view of the distal end 14 of the knot tying device 10 of the present invention and illustrating the end opening 22 and the transition portion 28 that leads into the side opening 18 (FIG. 3).

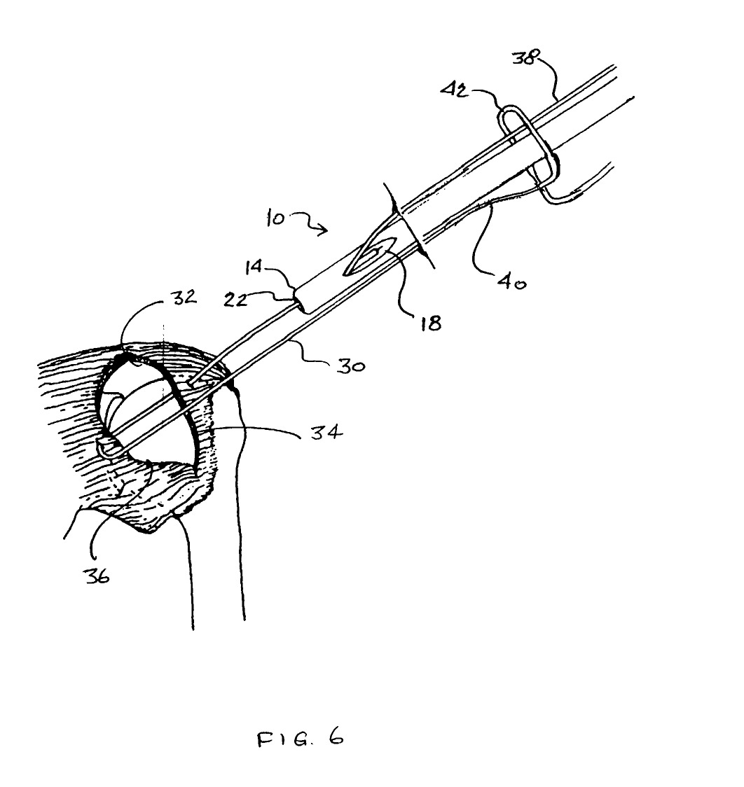

Next, turning now to FIG. 5, there is shown, a perspective view of the distal end 14 of the knot tying device 10 and illustrating the end opening 22. In this Figure there is also shown a suture strand 30 that has been passed through the end opening 22, the passageway 24 and has emerged through the side opening 18 where it is accessible by the physician to carry out the use of the knot tying device 10 as will be later explained.

The end opening 22 is designed to have a predetermined diameter “D” that is just slightly larger in dimensions than the diameter “d” of the suture strand 30 so that the suture strand 30 can freely move and slide through the end opening 22 but a larger body will not enter that end opening 22. For example, the end opening 22 may be a few thousandths of an inch larger than the suture strand 30 such that, as will be later clear, a knot tied in the suture strand 30 cannot pass thorough the end opening 22 and therefore cannot enter the passageway 24.

Of course, the exact predetermined dimensions of the diameter D of end opening 22 may depend upon the actual suture strand 30 and its dimensions since various suture strand sizes can be used by a physician, it only being of importance that the particular predetermined diameter D be slightly larger than the diameter “d” of the particular suture strand 30 that is being used for the suturing of the patient's tissue in order to carry out the method of the present invention using the knot tying device 10.

As also can be seen in FIG. 5, taken along with FIG. 4, however, the transition portion 28 of the passageway 24 opens or widens toward the side opening 18 to make easier, the threading of a suture strand 30 entering the end opening 22 and being passed through passageway 24 and outwardly through the side opening 18 where the suture strand 30 can be readily grasped by the physician.

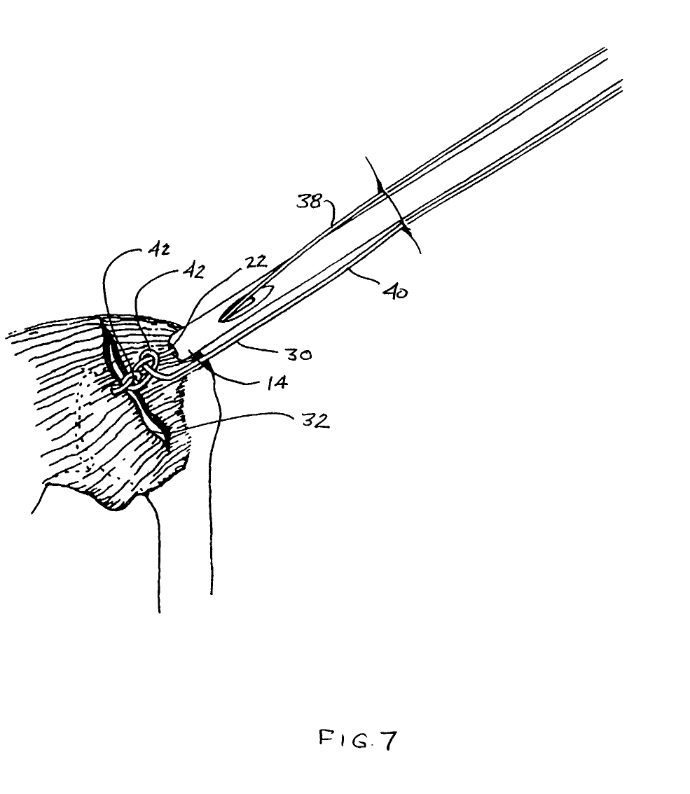

Turning now to FIG. 6, there is shown a schematic view of a knot tying device 10 of the present invention having a suture strand 30 interthreaded therein in order to illustrate the method of the present invention. Therefore, using a joint of a patient as an example, which may be a shoulder joint, there is a tear 32 in the tissue such that there is a forward edge 34 and a rearward edge 36 of that tear 32. The suture strand 30 has passed through the forward edge 34 of the tear 32 and been looped through the rearward edge 36 so that the suture strand has been passed through both the forward and rearward edges 34, 36, of the tear 32 in carrying out the suturing of the tear 32, that is, the bringing together of the forward and rearward edges 34, 36 to repair the tear 32.

As also can be seen, the suture strand 30 has first and second free ends 38, 40, one of which, for example, the first free end 38 has been brought through the distal end 14 of the knot tying device 10 by threading the first free end 38 into the end opening 22, through the passageway 24 (FIGS. 3 and 4) and outwardly through the side opening 18 and the physician therefore has access to the first free end 38 of the suture 30 that has passed through the knot tying device 10.

The other free end, the second free end 40 does not pass through the knot tying device 10 and can be used to form a knot, that is, the second free end 40 can form a loop 42 by the physician that passes around the first free end 38. That knot to be formed is basically a sliding knot as will be seen.

In FIG. 7, there is a schematic view of a later step in the use of the present inventive method and where a plurality of knots 42 have been formed to close the tear 32 in the tissue of the patient. In this step, the knot 42 or knots that thereby formed, are, of course, larger than the dimensions, or outer diameter of a single suture strand 30 and, therefore, the knots 42 cannot enter the end opening 22 since the end opening 22 has been predetermined dimensionally to be just slightly larger than the diameter of the suture strand 30 and thus a knot 42, being larger than a single suture strand 30 cannot enter the end opening. Accordingly, by the use of the knot tying device 10, a knot 42 can be slid toward the tear 32 along the second free end 40 so that a knot 42, can be slipped to a position where it is close to and tightly pushed against the now narrowed tear 32 in the tissue of the patient.

As such, the knot 42 is pushed toward the tear 32 by pulling or applying tension to the first free end 38 that passes through the knot tying device 10 and that knot 42 is thereafter tightened as additional tension is alternatively applied to the first and second free ends 38, 40 of the suture strand 30. During the tightening of the knot 42, the knot 42 is continuously held firmly against the tissue by the distal end 14 of the knot tying device 10 so that the knot 42 can be securely tightened while being held in the position against the tissue to join the tear 32 together.

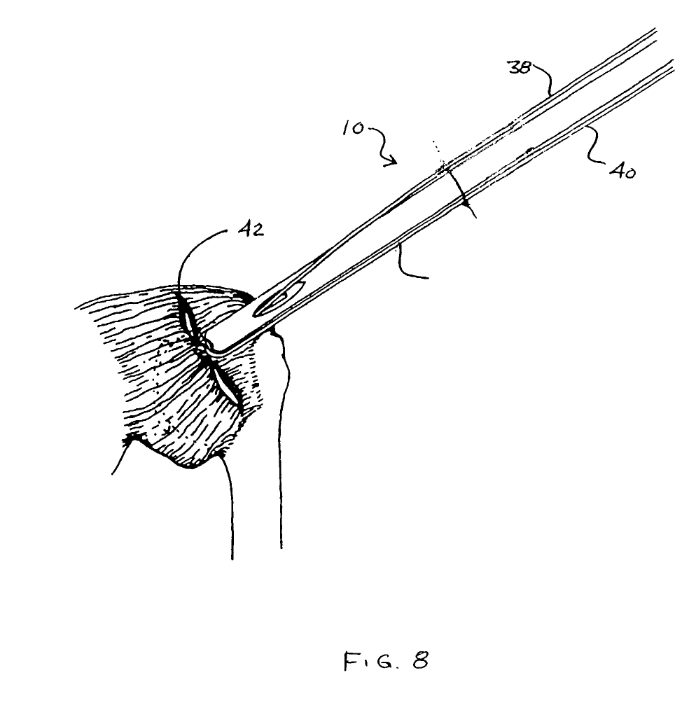

Finally, in FIG. 8, there is a schematic view of the knot 42 that has been pulled into a tight position. The knot 42, has been tightened in that position and held against the tissue of the patient by tension applied to the free ends 38, 40 of the suture strand 30 so that the final knot 42 holds the tear 32 together and the tear 32 has been repaired.

Those skilled in the art will readily recognize numerous adaptations and modifications which can be made to the knot tying device and method of using the same of the present invention which will result in an improved process and device, yet all of which will fall within the scope and spirit of the present invention as defined in the following claims. Accordingly, the invention is to be limited only by the following claims and their equivalents.

I claim:

1. A device for positioning a knot in suture strands tightly against tissue within a patient, said device comprising a main body having a central, elongated axis, a distal end and a proximal end, said distal end having an end opening generally in alignment with the elongated axis of the device, and a side opening in the side wall of the device with a passageway between said end opening and said side opening.

2. A device as defined in claim 1 wherein said side opening is located in close proximity to said distal end.

3. A device as defined in claim 2 wherein said side opening is located within about 2 cm. to about 4 cm. from said distal end.

4. A device as defined in claim 1 wherein said end opening has a predetermined diameter, said diameter being just slightly larger than the diameter of a single strand of suture.

5. A device as defined in claim 1 wherein said end opening has a diameter that is about one to ten thousandths of an inch larger than the diameter of a single strand of a suture.

6. A device as defined in claim 1 wherein the passageway has a coaxial portion extending coaxially along the longitudinal axis of the main body to a transition area located between the coaxial portion of the passageway and the side opening.

7. A device as defined in claim 6 wherein the transition area has an arcuate surface.

8. A device as defined in claim 7 wherein said transition area widens in the direction of the side opening.

9. A device as defined in claim 8 wherein said side opening is located about 2 to 4 centimeters from said distal end.

10. A device for positioning a knot in suture strands tightly against tissue within a patient, said device comprising a main body having a central, elongated axis, a distal end and a proximal end, said distal end having a rounded end surface and having an end opening generally in alignment with the elongated axis of the device, a side opening in the side wall of the device and a passageway between said end opening and said side opening, said passageway having a coaxial portion extending inwardly from said end opening for a predetermined distance terminating at an inward end and having a transition portion extending from the inward end of said coaxial portion to said side opening, said transition portion comprised of an arcuate solid surface within said device.

11. A device as defined in claim 10 wherein said transition portion of said passageway creates a bend of about ninety degrees in said passageway.

12. A device as defined in claim 11 wherein said transition portion widens in the direction toward said side opening.

13. A device as defined in claim 12 wherein said main body is comprised of a metal material.

14. A device as defined in claim 10 wherein said end opening is about one to ten thousandths of an inch larger than the diameter of a single suture strand.

15. A device as defined in claim 14 wherein said side opening is located about 2 to 4 centimeters from said distal end.

16. A method of tying a suture knot to join tissue together of a patient, said method comprising the steps of:
providing a device having a distal end, a proximal end and an exterior side surface, and wherein the device has an end opening at the distal end and a side opening in the exterior side surface with a passageway between the end opening and the side opening,
passing a suture strand through the tissue of a patient to provide two loose ends of the suture strand,
inserting one of the loose ends into the end opening and threading that loose end through the passageway to be accessible at the side opening of the device,
tying a knot between the loose ends,
pushing the device toward the tissue being sutured to move the knot into a location tightly pressed again the tissue, and
pulling on one or both of the loose ends of the suture strands to tighten the knot while holding the knot located in said position tightly pressed against that tissue.

17. A method as defined in claim 16 wherein said step of pulling on one of the loose ends of the suture strand comprises pulling on the loose end of the suture strand that has passed through the passageway.

18. A method as defined in claim 16 wherein said step of providing a device comprises providing a device having an end opening just slightly larger than a single suture strand.

19. A method as defined in claim 16 wherein said step of providing a device comprises providing a device wherein the passageway has a coaxial portion extending inwardly of the end opening for a predetermined distance coaxial with the longitudinal axis of the device and an arcuate transition portion that extends from said coaxial portion to the side opening.

20. A method as defined in claim 16 wherein said step of providing a device comprises providing a device wherein the transition portion widens in the direction of the side opening.
